(19) United States
(12) Patent Application Publication
(10) Pub. No.: US 2008/0033292 A1
(43) Pub. Date: Feb. 7, 2008

(54) ULTRASOUND PATIENT INTERFACE DEVICE

(75) Inventor: Yaron Shafran, Beit Hanania (IL)

Correspondence Address: VISTA IP LAW GROUP LLP, 12930 Saratoga Avenue, Suite D-2, Saratoga, CA, 95070, US

(73) Assignee: INSIGHTEC LTD, Tirat Carmel (IL)

(21) Appl. No.: 11/534,169

(22) Filed: Sep. 21, 2006

Related U.S. Application Data
(60) Provisional application No. 60/821,223, filed on Aug. 2, 2006.

(51) Int. Cl. A61B 8/00

(52) U.S. Cl. 600/437

(57) ABSTRACT

An ultrasound coupling device for acoustically coupling an ultrasound transducer with a patient's body comprises a fluid chamber having an ultrasound transducer interface for placing an ultrasound transducer in contact with water within said fluid chamber. The coupling device also has a patient interface comprising a water permeable membrane. The membrane has an internal membrane surface which is in contact with the water in the water tank and an external membrane surface can be placed in contact with the patient's body. The membrane is specially designed to allow water from inside the water tank to slowly permeate or “leak” to the external membrane surface while such surface is in contact or placed in very close proximity to the patient's skin. The slow permeation rate maintains the external membrane surface wet and free of air bubbles during a procedure which can take several hours. At the same time, the slow rate of permeation does not cause any water spillage around the patient.

### RELATED APPLICATIONS

This application claims the benefit of U.S. Provisional Patent Application Ser. No. 60/821,223, filed Aug. 1, 2006, pursuant to 35 U.S.C. Section 119, and any other applicable laws. The aforementioned application is hereby incorporated by reference herein in its entirety.

### FIELD OF THE INVENTION

The field of the invention generally relates to medical device which utilize ultrasound energy for performing medical procedures and diagnosis, and more particularly to a device for efficiently transmitting ultrasound energy between the ultrasonic device and the patient.

### BACKGROUND OF THE INVENTION

Ultrasound devices are widely used in the medical device field for many different applications. Ultrasound devices have many advantages, the foremost being the ability to image and access internal objects non-invasively. Also, they emit sonic energy which is relatively safe such as compared to x-ray radiation and other types of energy. In addition, ultrasound devices can be compact and relatively reasonable (compare to an MRI system).

The most common medical ultrasound devices are used for imaging. For example, ultrasonic imaging is commonly used to provide images of the internal tissue of a human body or other organism. Such ultrasonic imaging may be used for numerous medical procedures, including diagnosis, surgery, non-invasive surgery, minimally invasive surgery, and the like.

In addition to imaging, ultrasound devices are also known to be used for performing treatment on tissues. For instance, ultrasonic ablation instruments use focused ultrasonic energy for ablating tissue and other matter within a human body, such as fibroids, cancerous or non-cancerous tissue, breaking up occlusions within vessels or other matter such as kidney stones, and performing other treatments on or within a patient.

Generally, medical ultrasound devices utilize an electro-acoustic transducer within a housing. The housing typically has a “window” through which the transducer emits an ultrasound energy beam to an anatomical location. The device may incorporate lenses or other focusing elements to focus the energy to the desired size, magnitude and location. In use, the window is pressed against the skin of the patient's body and then the ultrasound device is operated.

However, the ultrasonic energy waves do not efficiently transmit from the window to the patient's body due to several factors. For one, there is not full and complete contact between window and the patient's body for which the ultrasound waves to travel. This further allows there to be air between the two surfaces which also inhibits the transmission of the ultrasonic waves. Furthermore, the interface between the window and the patient's skin creates an “impedance boundary,” i.e. a change in the impedance of the material through which the ultrasound waves travel. An impedance boundary tends to reflect and attenuate the ultrasonic energy.

In order to improve the transmission of the ultrasound energy from an ultrasound device into a patient's body, coupling agents have been employed. Existing coupling methods include gel pastes, molded gels, open water tanks or sealed water tanks. The coupling agents utilize water and other materials which have a similar speed of sound as human tissue in order to minimize ultrasound beam reflection a the interface and refraction. The coupling agent should also have low acoustic absorption and be as uniform as possible in order to minimize distortions. Good heat conduction properties are also an desirable because ultrasound devices (especially therapeutic focused ultrasound devices) create significant heat while being operated.

Ultrasound gel pastes have proven relatively useful as a coupling agent because they require minimum preparation and can be used on any shape and surface. However, gel pastes are messy and tend to capture air bubbles and are difficult to apply evenly. In addition, during long sessions, gel pastes tend to dry out thereby requiring re-application (including removal of the dried paste).

Molded gels are also a common and relatively convenient coupling agent. Molded gels provide good sound wave transmission and, unlike gel pastes, are not messy. But molded gels have several significant disadvantages. For one, molded gels do not conform well to the shapes of complex anatomical surfaces which results in poor surface contact. Moreover, gels are poor heat conductors and in certain cases may actually function as insulation which increases the temperatures experienced at the interface of the patient surface and the ultrasound device. As a result, molded gel devices may require complicated forced air/water cooling systems. This leads to a complex and cumbersome interface setup. More troubling, the gel and the skin may dry during the procedure which may not only negate the coupling effect of the device by may lead to partial or total blockage of the acoustic beam propagation.

Open water tanks provide excellent acoustic coupling because there is direct contact between the patient and the water. For the same reason, such tanks are excellent in keeping the interface wet and preventing the skin from drying. Also, open water tanks provide an ample heat sink for cooling the ultrasound device and it is fairly easy to control the temperature of the water. But open water tanks have serious shortcomings. Open water tanks do not easily support coupling from multiple directions because the water tank must be located directly beneath the body surface or the water will spill. Special sealing devices can be added to prevent spilling, but then it is difficult to couple to complex surfaces and shapes. Additionally, the water in the tank should remain degassed (free of air), but the tank is open, an inline degasser is needed during a procedure. There must also be a process for cleaning/sterilizing the water tank between patients. This typically entails draining the entire tank and sterilizing it.

Sealed water tanks, such as bags, balloons or pouches, are a convenient and clean device for ultrasonic coupling. Unlike water tanks, a sealed water device can support coupling from multiple directions. They are also very effective at conforming to complex anatomical surfaces and like water tanks, provide a good cooling of the interface and are easy to control thermally. However, sealed water devices must utilize a containment layer which does not provide as effective surface contact as an open water tank and may attenuate the acoustic energy at the interface. Sealed water devices also do not provide moisture to the body surface such that the surface tends to dry during the procedure.

Therefore, there is need for an ultrasound coupling device overcomes the problems associated with prior devices and agents.

### SUMMARY OF THE INVENTION

The present invention comprises a portable ultrasound patient interface device that provides an effective acoustic matching interface between an ultrasound transducer and a patient's body (typically the skin). The ultrasound coupling device comprises a fluid chamber having an ultrasound transducer interface for placing an ultrasound transducer in contact with water within said fluid chamber. The coupling device also has a patient interface comprising a water permeable membrane. The membrane has an internal membrane surface which is in contact with the water in the water tank and an external membrane surface can be placed in contact with the patient's body.

The membrane is specially designed to allow water from inside the water tank to slowly permeate or “leak” to the external membrane surface while such surface is in contact or placed in very close proximity to the patient's skin. The slow permeation rate maintains the external membrane surface wet and free of air bubbles during a procedure which can take several hours. At the same time, the slow rate of permeation does not cause any water spillage around the patient.

The membrane material is biocompatible for external use on a patient's body. Also, the membrane is preferably flexible such that it can conform to complex patient surfaces either by applying external pressure biasing the devices and the membrane into contact with the patient surface or by controlling the internal water pressure. Since the water is contained within the water tank, with only slow permeation through the membrane, the coupling device of the present invention can be used in any orientation without spilling the water within the tank. Additionally, the water tank can provide excellent heat dissipation because the water inside the tank can be cooled such as by free or forced convection or conduction.

Since the membrane is water permeable and actually contains water within its structure when in use, the membrane characteristics for sound transmission are similar to the water inside. Still, in order to minimize any distortion that might be caused by the membrane, the membrane should be as thin as possible, while still providing sufficient mechanical strength to support the water pressure behind the membrane and also providing the correct permeation rate. The water inside the tank can be degassed to minimize distortion that could be caused by air bubbles.

Accordingly, the ultrasound coupling device of the present invention effectively combines the advantages of molded gels with those of water tanks. Namely, the coupling device provides excellent sound transmission without the mess of gel pastes, can be used on body surfaces in any orientation without water spillage, effectively dissipates heat, can be easily controlled thermally, keeps the skin from drying out and maintains the water free from air bubbles during a procedure.

The ultrasound coupling device may be fully integrated with the ultrasound transducer device to provide a convenient, portable ultrasound apparatus having a built-in ultrasound coupling device. The ultrasound apparatus comprises a housing which is enclosed on all sides except for a large opening on the bottom side. An ultrasound transducer assembly comprising a plurality of ultrasound tiles is mounted within said housing with the ultrasound tiles oriented to transmit ultrasound energy through the opening in the housing. The opening in the housing is covered by the water permeable membrane as described above to form a water chamber enclosed by the housing and the membrane. The housing has two water ports in fluid communication with the water chamber for filling, draining and otherwise conditioning the water in the water chamber.

The ultrasound apparatus may further comprise a ring-like membrane mounting flange around the opening in the housing onto which the membrane is mounted. A membrane locking ring mounts onto the mounting flange over the membrane to secure the membrane between the mounting flange and the locking ring. The water ports may be conveniently provided on the mounting flange of the housing.

### BRIEF DESCRIPTION OF THE DRAWINGS

FIG. 1 is a side perspective view of an ultrasound apparatus having an ultrasound coupling device, according to the present invention.

FIG. 2 is a side perspective, exploded view of the ultrasound apparatus of FIG. 1.

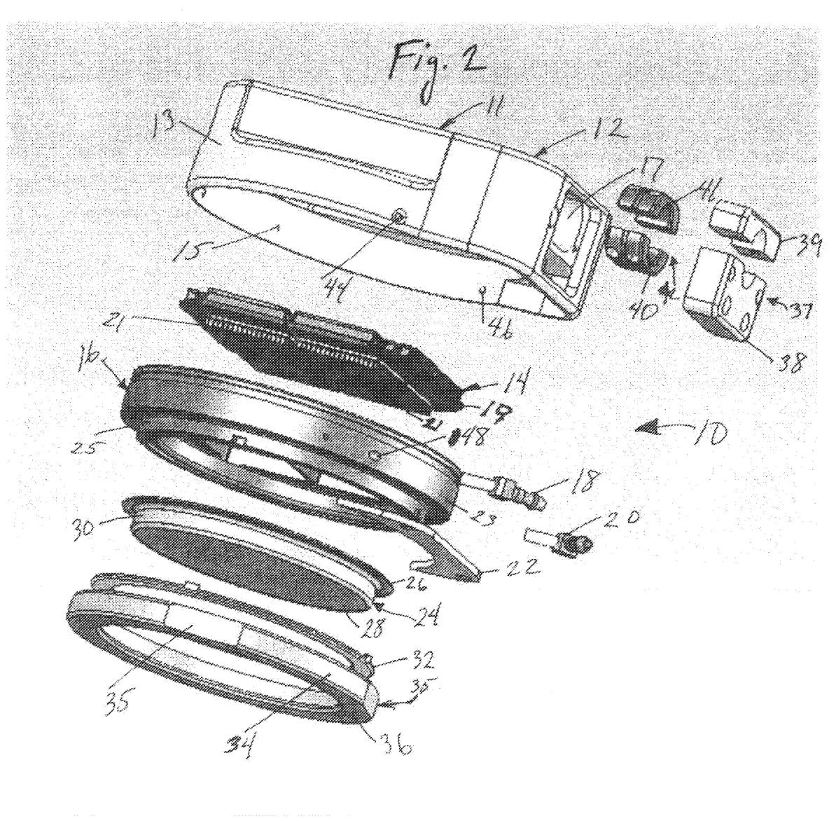

### DETAILED DESCRIPTION OF THE INVENTION

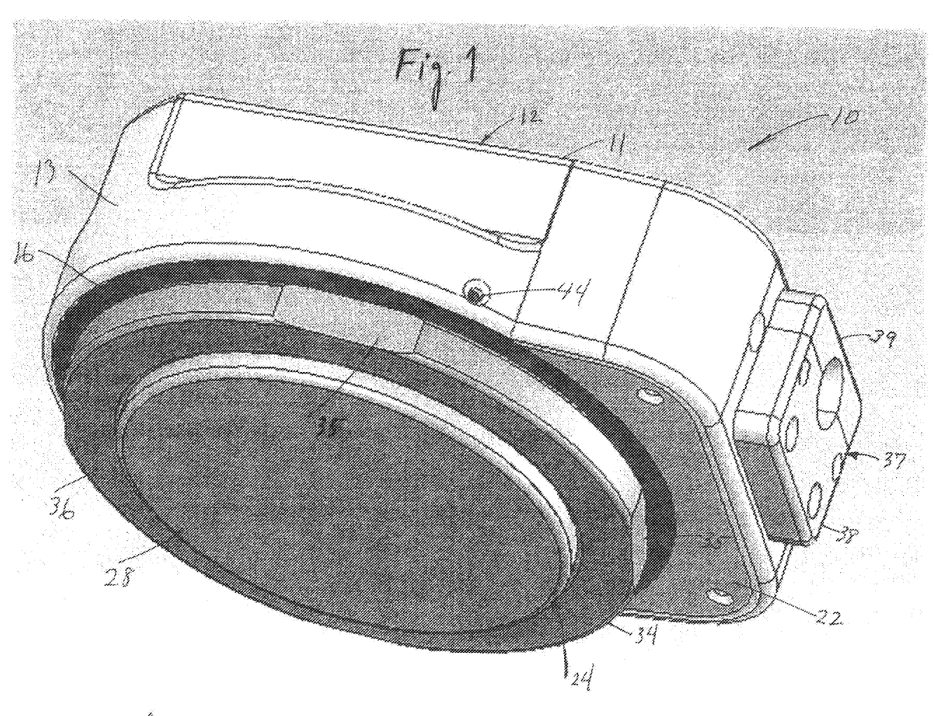

As shown in FIG. 1 the ultrasound apparatus with ultrasound coupling device 10 according to the present invention comprises a main housing 12. The main housing 12 has a closed top 11, a sidewall 13 extending from the top 11 and a large opening 15 opposite the top 11. As will be understood by those of skill in the art, the devices and structures described herein may be used substantially the same regardless of their absolute position or orientation relative to external axes or objects. Accordingly, terms having directional meanings such as top, bottom, upper, lower, left and right as used herein refer only to the relative locations of elements and do not refer, or limit the invention or its elements, to any particular absolute orientation with respect to any external axes or objects, unless the context clearly dictates otherwise.

Although the ultrasound apparatus 10 and the major components like the membrane 24 and mounting flange 16 are shown as being generally round, it is to be understood that the apparatus 10 and its components are not to be limited to such shape. It should be readily appreciated that the apparatus 10 can be of any suitable shape, such as any polygonal shape, oval, elliptical, and other curved shapes.

The sidewall 13 of the housing 12 has a cable opening 17 for running cables or other hardware into the housing 12. A cable bracket 40 is provided in the cable opening for securing and/or sealing the cables. The cable bracket 42 has an upper portion 41 and lower portion 42 which are bolted together. An o-ring is seated in the outside groove of the bracket 42 and sealing glue is applied to seal any space between the cables and the opening in the bracket 42. A securing bracket 37 having an upper portion 39 and a lower portion 38 mounts over the cable bracket and over the cable opening 17. An additional opening beneath the opening 17 is used for a pair of water inlet/outlet hoses 18 and 20.

An ultrasound transducer assembly 14 is mounted within the housing 12. The ultrasound transducer assembly may have a substantially flat mounting plate 19 upon which a plurality of ultrasound transducer tiles 21 are mounted. With the transducer assembly installed, the flat surface of the mounting plate 19 faces the opening 15 and the transducer tiles 21 are oriented to transmit ultrasound energy through the membrane 30. The mounting plate 19 is mounted to a mounting surface (not shown) in the housing 12. The interface between the mounting surface and mounting plate 19 may provide a water-tight seal. A gasket or other sealing device may be used to provide the water-tight seal.

A membrane mounting flange 16 fits into and/or around the opening 15 in the housing 12. The membrane mounting flange 16 has a main ring portion 23 and a raised lip portion 25 extending from said ring portion 23 onto which the membrane 24 is mounted. The raised lip portion 25 defines an opening in the flange 16 which is covered by the membrane. A retaining bracket 22 bolts onto the housing 12 to cover the hoses 18 and 20. The mounting flange 16 is secured to the housing 12 using an o-ring disposed in a mounting flange groove and several mounting screws (in 44, 46 etc.). The membrane mounting flange 16 has a pair of water inlet/outlet holes 48 which are in fluid communication with the hoses 18 and 20 and the holes in the lower portion 38 of the cable bracket 37.

The membrane 24 has a main surface 28 which is substantially spheroidal shape and which covers the opening defined by the raised lip portion 25 of the mounting flange 16. The membrane 24 also has a membrane sidewall 30 extending axially from the main surface 28 and a membrane flange 26 extending radially from the membrane sidewall 30. At least the main surface of the membrane is formed of a water permeable material which allows water to slowly permeate or “leak” through the mains surface 28 such that the external surface of the main surface 28 remains wet and free of air bubbles during the use of the apparatus 10. Because the membrane is in contact with a patient's body during use of the apparatus 10, the membrane material is biocompatible for external use on a patient's body. The membrane material is also flexible so that it can conform complex patient surfaces when the apparatus 10 is in use.

Suitable materials for the membrane 24 include Tecophilic® TPUs (thermoplastic polyurethanes). These materials and films formed from these materials are available from Noveon, Inc. (formerly Thermedics) in Cleveland, Ohio. Persons of ordinary skill in the art, and in view of this specification, will understand how to properly configure and manufacture the membrane 24 to function as described herein. The membrane should be as thin as possible, while still providing sufficient mechanical strength to support the water pressure and to avoid being damaged under normal conditions of use and also providing the correct permeation rate to keep the outer surface wet. The membrane 24 has a thickness of about 0.2 mm to 0.4 mm, or alternatively less than 0.4 mm, or alternatively 0.15 mm to 0.45 mm.

A membrane locking ring 36 mounts to the mounting flange 16 and over the membrane 24 to secure the membrane 16. The locking ring 36 has a retaining surface 36 which is substantially parallel to the main surface 28 of the membrane 16. A ring sidewall 34 extends axially from the retaining surface 36. The ring sidewall 34 has a plurality of flat installation surfaces 35 for facilitating the installation of the locking ring 36 to the mounting flange 16. The locking ring 36 can be secured to the mounting flange 16 by mating threads, bolts or other suitable mounting means. A gasket 32 may be provided between the locking ring 36 and the mounting flange 16 to help provide a water-tight seal between the components.

The membrane 24 and/or the may be made to be disposable so that the apparatus 10 does not need to be cleaned and sterilized between uses. Between uses, the membrane 24 is simply removed from the apparatus 10 by removing the locking ring 36. A new membrane 24 is installed and the locking ring 36 is re-installed.

A pair of inlet/outlet water ports 18 and 20 connect to the inlet/outlet water ports 48 (the port on the back side of 16 is hidden from view in the figures) on the sidewall 13 of the housing 12. The water ports 18 and 20 may be connected to a water conditioning apparatus which can control various conditions of the water, such as the water pressure and/or temperature, and may also degas the water.

Although the apparatus 10 has been shown and described with a generally circular shape and circular membrane 24, it should be understood that the apparatus 10 can have any desired shape. For instance, the transducer assembly 14 can be shaped for a specific anatomical region such as transvaginal or transrectal imaging. In such case, the apparatus 10 and each of the components described above can be appropriately shaped to imaging in these certain anatomical regions of the body.

While embodiments of the present invention have been shown and described, various modifications may be made without departing from the scope of the present invention. The invention, therefore, should not be limited, except to the following claims, and their equivalents.

We claim:

1. An ultrasound patient interface device for coupling an ultrasound transducer with a patient's body, the device comprising:
a fluid chamber having an ultrasound transducer interface for placing an ultrasound transducer in contact with water within said fluid chamber; and
a water permeable membrane having an internal membrane surface in contact with said water within said fluid chamber, and an external membrane surface which can be placed in contact with the patient's body, said membrane being water permeable to allow said water within the fluid chamber to slowly permeate said membrane to wet said external membrane surface but does not allow said water to flow out of said fluid chamber.

2. The ultrasound patient interface device of claim 1, wherein said fluid chamber comprises a housing having a bottom, a sidewall extending from said bottom and defining an opening, and wherein said membrane covers said opening.

3. The ultrasound patient interface device of claim 1, further comprising:
a housing having a bottom and a sidewall extending from said bottom and defining an opening opposite said bottom, said sidewall defining at least a portion of said fluid chamber which is open at said opening, and
a membrane mounting flange disposed on said housing about said opening, said membrane mounting flange having a main ring portion and a raised flange extending from said ring portion upon which said membrane is mounted.

4. The ultrasound patient interface device of claim 3, wherein a bottom side of said fluid chamber is defined by said bottom.

5. The ultrasound patient interface device of claim 3, wherein at least a portion of a bottom side of said fluid chamber is defined by a structure within said housing, at least a portion of said structure extending to said sidewall.

6. The ultrasound patient interface device of claim 3, wherein a bottom side of said fluid chamber is defined by an ultrasound transducer mounting plate mounted within said housing, said ultrasound transducer mounting plate having a plurality of ultrasound transducers mounted thereon.

7. The ultrasound patient interface device of claim 3 further comprising a locking ring which mounts to said mounting flange over said membrane thereby securing said membrane between said locking ring and said mounting flange.

8. An ultrasound apparatus, comprising:
a housing having a bottom and a sidewall extending from said bottom and defining an opening opposite said bottom, said sidewall defining at least a portion of a fluid chamber for containing water which is open at said opening, said housing having a membrane mounting flange disposed at said opening;
an ultrasound transducer mounted within said fluid chamber; and
a water permeable membrane mounted on said mounting flange, said membrane having an internal membrane surface in contact with said water within said fluid chamber, and an external membrane surface which can be placed in contact with the patient's body, said membrane being water permeable to allow said water within the fluid chamber to slowly permeate said membrane to wet said external membrane surface but does not allow said water to flow out of said fluid chamber.

9. The apparatus of claim 8, wherein said membrane mounting flange comprises a main ring portion and a raised flange extending from said ring portion upon which said membrane is mounted.

10. The apparatus of claim 8, wherein said membrane mounting flange is formed integrally with said housing.

11. The apparatus of claim 8, wherein said membrane mounting flange is a separate part attached to said housing.

12. The apparatus of claim 8 further comprising a locking ring which mounts to said mounting flange over said membrane thereby securing said membrane between said locking ring and said mounting flange.

13. The apparatus of claim 8, wherein said ultrasound transducer is mounting on an ultrasound transducer mounting plate, said mounting plate forming defining a least a portion of said fluid chamber.
